(19) United States
(12) Patent Application Publication
(10) Pub. No.: US 2025/0144195 A1
(43) Pub. Date: May 8, 2025

(54) CHIMERIC ANTIGEN RECEPTOR-EXPRESSING T CELLS AS ANTI-CANCER THERAPEUTICS

(71) Applicant: Purdue Research Foundation, West Lafayette, IN (US)

(72) Inventors: Philip S. LOW, West Lafayette, IN (US); Haiyan Chu, West Lafayette, IN (US); Yong Gu Lee, West Lafayette, IN (US)

(21) Appl. No.: 18/915,595

(22) Filed: Oct. 15, 2024

Related U.S. Application Data
(63) Continuation of application No. 17/387,892, filed on Jul. 28, 2021, now Pat. No. 12,150,981.
Application No. 17/387,892 is a continuation of application No. 15/296,666, filed on Oct. 18, 2016, now abandoned.
Application No. 15/296,666 is a continuation of application No. 14/654,227, filed on Jun. 19, 2015, now abandoned.
(60) Provisional application No. 61/740,384, filed on Dec. 20, 2012.

(51) Int. Cl. A61K 39/00; A61K 35/17; A61K 40/11; A61K 40/31; A61K 47/54; A61K 47/55; A61K 47/60; A61K 47/69

(52) U.S. Cl. CPC A61K 39/0013; A61K 35/17; A61K 40/11; A61K 40/31; A61K 47/545; A61K 47/551; A61K 47/555; A61K 47/60; A61K 47/6901; A61K 2039/5156; A61K 2039/5158; A61K 2039/585; A61K 2239/13

(57) ABSTRACT

Cytotoxic lymphocytes expressing chimeric antigen receptors (CAR) that target and bind small conjugate molecules (SCM) are disclosed, as well as methods of using the cells and the SCMs in the treatment of cancer.

### CROSS-REFERENCE TO RELATED APPLICATIONS

This application is a continuation of U.S. patent application Ser. No. 17/387,892, filed on Jul. 28, 2021, which is a continuation of U.S. patent application Ser. No. 15/296,666, filed on Oct. 18, 2016, which is a continuation of U.S. patent application Ser. No. 14/654,227, filed on Jun. 19, 2015, which is the national stage entry of International Patent Application No. PCT/US2013/076986, filed on Dec. 20, 2013, which claims benefit of priority to U.S. Provisional Application no. 61/740,384, filed on Dec. 20, 2012, each of which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is UMOJ_026_04US_ST25. The text file is 14 KB, created on Sep. 23, 2021, and is being submitted electronically via EFS-WEB.

### BACKGROUND

Immunotherapy based on adoptive transfer of lymphocytes (e.g., T cells) into a patient can play an important role in eliminating cancer. Among many different types of immunotherapeutic agents, one of the most promising therapeutic methods being developed is T cells expressing Chimeric Antigen Receptors (CAR). CARs are genetically engineered receptors that are designed to target a specific antigen of a selected tumor [1]. For example, T cells that have cytotoxic activity are transfected with and grown to express CARs such that T cells expressing CARs can target and kill tumors via tumor-associated antigens.

First generation CARs are composed of two main regions. First, a recognition region, e.g., a single chain fragment variable (scFv) region derived from a tumor-targeted antibody, is used to recognize and bind tumor-associated antigens. Second, an activation signaling domain, e.g., the CD3ζ chain of T cells, serves as a T cell activation signal in CARs [2]. Although T cells transduced to express such constructs showed positive results in vitro, they have been found to have limited performance in eliminating tumor cells in clinical trials. The main limitation has been the relative inability to prolong and expand the T cells population and achieve sustained antitumor effects in vivo.

To address these problems, a co-stimulation domain (e.g. CD137, CD28 or CD134) is included in second generation CARs to achieve full, prolonged activation of T cells. Addition of a co-stimulation domain enhances the in vivo proliferation and survival of T cells containing CARs, and initial clinical data have shown that such constructs are a promising therapeutic agent in the treatment of tumors [3].

Although use of CAR-expressing T cells as an immunotherapeutic agent shows promise, there remain several challenges to overcome in order to achieve significant clinical outcomes. First, ‘off-target’ toxicities may result due to the fact that it is difficult to target only cancer cells via tumor-associated antigens because in many cases normal cells also express the tumor-associated antigen. For example, CD19 is a tumor-associated antigen that is expressed on malignant B cells. CARs containing anti-CD19 antibody were generated and used treated to patients. Although remission of malignant B cells was found, normal B cells were depleted in the patients as well because normal B cells also express CD19 [4]. Another example pertains to carbonic anhydrase IX (CAIX) which is overexpressed in clear cell renal carcinoma. Liver toxicity was found in subjects of the first clinical trial using CAR-targeting CAIX, likely due to the fact that CAIX is also expressed in bile duct epithelial cells and as such, the T cells targeted normal tissue as well [5].

Second, ‘unregulated CAR activity’ may be found where the rapid eradication of cancer cells by CARs induces a constellation of metabolic disturbances, called tumor lysis syndrome or a cytokine storm, which can be a fatal consequence to patients [1, 4, 6]. This is a result because transduced T cells expressing CARs cannot be easily regulated. Once transduced T cells are infused to patients, it is currently very difficult to regulate or control the activation of the cells.

Therefore, while CAR-expressing T cells show great promise as a tool in the treatment of cancer, the next generation of the CAR system is needed that provides reduced off-target toxicity and greater control of activation. The present invention is directed to this and other important ends.

### BRIEF SUMMARY

The invention relates to a Chimeric Antigen Receptor (CAR) system and methods for using the system in the treatment of subjects with cancer. The CAR system of the present invention includes cytotoxic lymphocytes expressing CARs that target a moiety that is not produced or expressed by cells of the subject being treated. This CAR system thus allows for focused targeting of the cytotoxic lymphocytes to target cells, such as cancer cells. The targeted moiety is part of a small conjugate molecule (SCM) that also comprises a ligand of a tumor cell receptor. Administration of a SCM along with the CAR-expressing cytotoxic lymphocytes results in the targeting of the cytotoxic lymphocyte response to only those cells expressing the tumor receptor to which the SCM is bound.

In a first embodiment, the invention is directed to CAR-expressing cytotoxic lymphocytes. The CAR is a fusion protein comprising a recognition region, at least one co-stimulation domain, and an activation signaling domain. The CAR has binding specificity for a selected targeted moiety or can be bound by a targeted moiety.

In certain aspects of this embodiment, the recognition region of the CAR is a single chain fragment variable (scFv) region of an antibody with binding specificity for the targeted moiety. In a particular aspect, the recognition region of the CAR is a single chain fragment variable (scFv) region of an anti-FITC antibody.

In certain aspects of this embodiment, the co-stimulation domain of the CAR is selected from the group consisting of CD28, CD137 (4-1BB), CD134 (OX40), and CD278 (ICOS).

In certain aspects of this embodiment, the activation signaling domain of the CAR is the T cell CD3ζ chain or Fc receptor γ.

In certain aspects of this embodiment, the cytotoxic lymphocytes are one or more of cytotoxic T cells, natural killer (NK) cells, and lymphokine-activated killer (LAK) cells.

In a particular aspect of this embodiment, the recognition region is a single chain fragment variable (scFv) region of an anti-FITC antibody, the co-stimulation domain is CD137 (4-1BB), and the activation signaling domain is the T cell CD3ζ chain.

In certain aspects of this embodiment, the targeted moiety is a molecule selected from the group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

In certain aspects of this embodiment, the binding specificity of the CAR for the targeted moiety has an affinity of at least about 100 pM.

In a second embodiment, the invention is directed to small conjugate molecules (SCM) comprising a targeted moiety conjugated to a tumor receptor ligand, wherein the tumor receptor ligand is folate, 2-[3-(1,3-dicarboxypropyl)ureido] pentanedioic acid (DUPA), or cholecystokinin 2 receptor (CCK2R) ligand.

Targeted moieties that may be used in the SCMs of the invention include, but are not limited to, 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

In certain aspects of this embodiment, the targeted moiety and the ligand are conjugated via a linker domain. Linker domains include, but are not limited to, polyethylene glycol (PEG), polyproline, a hydrophilic amino acid, a sugar, an unnatural peptideoglycan, polyvinylpyrrolidone, and pluronic F-127. In a particular aspect, the linker domain is (PEG)₁₂.

In certain aspects of this embodiment, the targeted moiety is FITC.

In certain aspects of this embodiment, the targeted moiety is FITC and the linker is (PEG)₁₂.

In particular aspects of this embodiment, the SCM is FITC-folate, FITC-DUPA, FITC-CCK2R ligand, FITC-(PEG)₁₂-folate, FITC-(PEG)₁₂-DUPA, or FITC-(PEG)₁₂-CCK2R ligand.

In a third embodiment, the invention is directed to a two component cancer therapeutic comprising:
(a) a small conjugate molecule (SCM) comprising a targeted moiety conjugated to a tumor receptor ligand, wherein the tumor receptor ligand is folate, DUPA, or CCK2R ligand; and
(b) chimeric antigen receptor (CAR)-expressing cytotoxic lymphocytes, wherein the CAR is a fusion protein comprising a recognition region, a co-stimulation domain and an activation signaling domain, and wherein the CAR has binding specificity for the targeted moiety.

Targeted moieties that may be used in the SCMs include, but are not limited to, 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

In certain aspects of this embodiment, the targeted moiety and the ligand are conjugated via a linker domain. Linker domains include, but are not limited to, polyethylene glycol (PEG), polyproline, a hydrophilic amino acid, a sugar, an unnatural peptideoglycan, polyvinylpyrrolidone, and pluronic F-127. In a particular aspect, the linker domain is (PEG)₁₂.

In certain aspects of this embodiment, the targeted moiety is FITC.

In certain aspects of this embodiment, the targeted moiety is FITC and the linker is (PEG)₁₂.

In particular aspects of this embodiment, the SCM is FITC-folate, FITC-DUPA, FITC-CCK2R ligand, FITC-(PEG)₁₂-folate, FITC-(PEG)₁₂-DUPA, or FITC-(PEG)₁₂-CCK2R ligand.

In certain aspects of this embodiment, the recognition region of the CAR is a single chain fragment variable (scFv) region of an antibody with binding specificity for the targeted moiety. In a particular aspect, the recognition region of the CAR is a single chain fragment variable (scFv) region of an anti-FITC antibody.

In certain aspects of this embodiment, the co-stimulation domain of the CAR is CD28, CD137 (4-1BB), CD134 (OX40), or CD278 (ICOS).

In certain aspects of this embodiment, the activation signaling domain of the CAR is the T cell CD3ζ chain or Fc receptor γ.

In certain aspects of this embodiment, the cytotoxic lymphocytes are one or more of cytotoxic T cells, natural killer (NK) cells, and lymphokine-activated killer (LAK) cells.

In a particular aspect of this embodiment, the recognition region is a single chain fragment variable (scFv) region of an anti-FITC antibody, the co-stimulation domain is CD137 (4-1BB), and the activation signaling domain is the T cell CD3ζ chain.

In certain aspects of this embodiment, the binding specificity of the CAR for the targeted moiety is an affinity of at least about 100 pM.

In a fourth embodiment, the invention is directed to a method of treating cancer in a subject. In a first aspect the method comprises:
(a) culturing a population of cytotoxic lymphocytes under conditions promoting activation;
(b) transfecting the lymphocyte population of (a) with a vector encoding a chimeric antigen receptor (CAR), wherein the CAR is a fusion protein comprising a recognition region, a co-stimulation domain and an activation signaling domain;
(c) administering a therapeutically effective number of the transfected lymphocytes of (b) to a subject having cancer; and
(d) administering a small conjugate molecule (SCM) comprising a targeted moiety conjugated to a tumor receptor ligand to the subject, wherein the ligand is recognized and bound by a receptor on the surface of a cell of the cancer, and wherein the CAR has binding specificity for the targeted moiety or can be bound by the targeted moiety;
thereby treating cancer in a subject.

In a related embodiment the method comprises:
(a) culturing a population of cytotoxic lymphocytes under conditions promoting activation;
(b) transfecting the lymphocyte population of (a) with a vector encoding a chimeric antigen receptor (CAR), wherein the CAR is a fusion protein comprising a recognition region, a co-stimulation domain and an activation signaling domain;
(c) administering a small conjugate molecule (SCM) comprising a targeted moiety conjugated to a tumor receptor ligand to a subject having cancer, wherein the ligand is recognized and bound by a receptor on the surface of a cell of the cancer; and
(d) administering a therapeutically effective number of the transfected T cells of (b) to the subject, and wherein the CAR has binding specificity for the targeted moiety or can be bound by the targeted moiety;
thereby treating cancer in a subject.

In a further related embodiment the method comprises:
(a) culturing a population of cytotoxic lymphocytes under conditions promoting activation;
(b) transfecting the lymphocytes population of (a) with a vector encoding a chimeric antigen receptor (CAR), wherein the CAR is a fusion protein comprising a recognition region, a co-stimulation domain and an activation signaling domain, and wherein the CAR has binding specificity for a targeted moiety or can be bound by the targeted moiety;
(c) incubating the lymphocytes of (b) with a small conjugate molecule (SCM) comprising a targeted moiety conjugated to a tumor receptor ligand;
(d) administering a therapeutically effective number of the transfected lymphocytes of (c) to a subject having cancer;
thereby treating cancer in a subject.

In these three related embodiments the cytotoxic lymphocytes may be autologous or heterologous cells, with respect to the subject being treated, or a combination of both.

In these three related embodiments the culturing conditions of (a) may comprise culturing the population of lymphocytes in the presence of anti-CD3 antibodies or anti-CD28 antibodies, or both.

In certain aspects of these three related embodiments, the recognition region of the CAR is a single chain fragment variable (scFv) region of an antibody with binding specificity for the targeted moiety. In a particular aspect, the recognition region of the CAR is a single chain fragment variable (scFv) region of an anti-FITC antibody.

In certain aspects of these three related embodiments, the co-stimulation domain of the CAR is CD28, CD137 (4-1BB), CD134 (OX40), or CD278 (ICOS).

In certain aspects of these three related embodiments, the activation signaling domain of the CAR is the T cell CD3ζ chain or Fc receptor γ.

In certain aspects of these three related embodiments, the cytotoxic lymphocytes are one or more of cytotoxic T cells, natural killer (NK) cells, and lymphokine-activated killer (LAK) cells.

In certain aspects of these three related embodiments, the recognition region is a single chain fragment variable (scFv) region of an anti-FITC antibody, the co-stimulation domain is CD137 (4-1BB), and the activation signaling domain is the T cell CD3ζ chain.

In certain aspects of these three related embodiments, the targeted moiety is a molecule selected from the group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin. In a particular aspect, the targeted moiety is FITC.

In certain aspects of these three related embodiments, the ligand is folate, DUPA, CCK2R ligand.

In certain aspects of these three related embodiments, the targeted moiety and the ligand are conjugated via a linker domain. The linker domain may be, for example, polyethylene glycol (PEG), polyproline, a hydrophilic amino acid, a sugar, an unnatural peptideoglycan, polyvinylpyrrolidone, or pluronic F-127.

In certain aspects of these three related embodiments, the targeted moiety is FITC and the linker is (PEG)₁₂.

In certain aspects of these three related embodiments, the vector is a lentivirus vector.

In certain aspects of these three related embodiments, the binding specificity of the CAR for the targeted moiety is an affinity of at least about 100 pM.

In certain aspects of these three related embodiments, the subject is a human.

In certain embodiments of these three related embodiments, the cancer is one or more of a cancer of the brain, thyroid, lung, pancreas, kidney, stomach, gastrointestinal stroma, endometrium, breast, cervix, ovary, colon, prostate, leukemias, lymphomas, other blood-related cancers or head and neck cancer.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1A is a schematic showing the CAR4-1BBZ construct.

FIG. 1B shows the transduction efficiency into T cells of the CAR4-1BBZ construct. 96 h after transduction, the expression of CAR4-1BBZ was identified through copGFP expression by flow cytometry. As shown in the figure, approximately 30% of transduced T cells expressed CAR4-1BBZ.

FIG. 1C shows the expression of CAR4-1BBZ on transduced T cells using confocal microscopy. copGFP was expressed on positive transduced T cells containing CAR4-1BBZ (arrows). However, copGFP expression was not detected on non-transduced T cells that do not express CAR4-1BBZ.

FIG. 2 shows binding of FITC-Folate and FITC-(PEG)12-Folate conjugates to CAR-transduced T cells. FIG. 2 shows confocal microscopy of copGFP expression in transduced T cells containing CAR 4-1BBZ (top row) and FITC-folate binding on CAR-1BBZ transduced T cells (middle row). The bottom row shows the same view in the absence of fluorescence.

FIGS. 3A-3D show the binding ability of FITC-Folate conjugates to cancer cells via florescence microscopy. FIG. 3A shows the binding of FITC-Folate conjugates to KB cancer cells. FIG. 3B shows the binding of FITC-Folate conjugates to L1210A cancer cells. FIG. 3C shows the binding of FITC-(PEG)₁₂-Folate conjugates to KB cancer cells. FIG. 3D shows the binding of FITC-(PEG)₁₂-Folate conjugates to L1210A cancer cells. The competitor was 50× excess folate acid.

FIGS. 4A-4B show the results of assays to determine whether CAR-expressing T cells are cytotoxic to cancer cells in the presence or absence of FITC-Folate conjugates using KB cells (FIG. 4A) or L1210A cells (FIG. 4B).

FIG. 5 shows an illustrative synthetic scheme.

FIG. 6 shows an illustrative synthetic scheme.

FIG. 7 shows an illustrative synthetic scheme.

FIG. 8 shows an illustrative synthetic scheme.

FIG. 9 shows an illustrative synthetic scheme.

### DETAILED DESCRIPTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, “treat” and all its forms and tenses (including, for example, treat, treating, treated, and treatment) refer to both therapeutic treatment and prophylactic or preventative treatment.

### II. The Present Invention

The present invention is directed to a CAR system for use in the treatment of subjects with cancer. The CAR system of the present invention (e.g., cytotoxic lymphocytes expressing novel CARs and cognate small conjugate molecules (SCM)) makes use of CARs that target a moiety that is not produced or expressed by cells of the subject being treated. This CAR system thus allows for focused targeting of the cytotoxic lymphocytes to target cells, such as cancer cells. The targeted moiety is part of a small conjugate molecule (SCM) that also comprises a ligand of a tumor cell receptor. Because small organic molecules are typically used as the targeted moiety, clearance of the SCM from the bloodstream can be achieved within about 20 minutes. By administration of a SCM along with the CAR-expressing cytotoxic lymphocytes, the lymphocyte response can be targeted to only those cells expressing the tumor receptor, thereby reducing off-target toxicity, and the activation of lymphocytes can be more easily controlled due to the rapid clearance of the SCM. As an added advantage, the CAR-expressing lymphocytes can be used as a “universal” cytotoxic cell to target a wide variety of tumors without the need to prepare separate CAR constructs. The targeted moiety recognized by the CAR may also remain constant. It is only the ligand portion of the SCM that needs to be altered to allow the system to target cancer cells of different identity.

One embodiment of the invention provides an illustration of this novel CAR system. In this embodiment, and as a first component, a SCM is prepared that comprises FITC linked to a ligand of a selected tumor cell receptor. As a second component, cytotoxic T cells are transduced to express a CAR that comprises anti-FITC scFv. This CAR thus targets Fluorescein Isothiocyanate (FITC) instead of a tumor-associated antigen that might also be expressed by healthy, non-target cells. The two components are administered to a subject having cancer and the FITC-SCM (first component) is bound by the target tumor cells (through binding of the ligand portion of the molecule to cognate tumor cell receptor). The FITC portion of the SCM is then recognized and bound by the anti-FITC CAR expressed by the T cells (second component). Upon FITC binding, the anti-FITC CAR-expressing T cells are activated and the tumor cell is killed. As will be apparent to the skilled artisan, the cytotoxic T cells cannot kill cells with first binding to a tumor cell. As it will be further apparent, T cells will not bind to non-target cells because the recognition region of the CAR will only recognize and bind FITC, which is not produce or expressed by cells of the subject. The SCM thus acts as a bridge between the cytotoxic T cells and the target tumor cells. As long as the targeted moiety of the SCM is a moiety not found in the host, the activity of the T cells can be limited to the target cells. Further, the activation of the CAR-expressing T cells can be regulated by limiting the amount of SCM administered to a subject, for example, by manipulating infusion of the small conjugate molecule if a side effect is detected. Thus, the CAR system of the present invention overcomes problems associated with conventional CAR therapy.

### Small Conjugate Molecules (SCM)

The CAR system of the present invention utilizes small conjugate molecules (SCMs) as the bridge between cytotoxic lymphocytes and targeted cancer cells. The SCMs are conjugates comprising a targeted moiety on one end of the molecule and a tumor receptor ligand on the other, optionally connected by a bridge domain. The targeted moiety is a molecule that is recognized by a CAR of a transduced lymphocyte or that can bind to a region of the CAR. The identity of the targeted moiety is limited only in that it must be a molecule that can be recognized and bound by CAR expressed by a lymphocyte, or recognized and bind the CAR itself, in both cases preferably with specificity, and that it have a relatively low molecular weight. Exemplary targeted moieties are haptens that can be recognized and bound by CARs and include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives. Suitable targeted moieties that themselves bind to one or more regions of a CAR include knottins [16], centyrins and DARPins [7].

The tumor receptor ligands that comprise the SCMs of the present invention are molecules recognized and bound by receptors expressed by target tumor cells, typically expressed on the surface of the tumor cells. Suitable ligands include: 1) DUPA (DUPA-(99 m)Tc), a ligand bound by PSMA-positive human prostate cancer cells with nanomolar affinity (K_{D}=14 nM; [8]); 2) CCK2R ligand, a ligand bound by CCK2R-positive cancer cells (e.g., cancers of the thyroid, lung, pancreas, ovary, brain, stomach, gastrointestinal stroma, and colon; [9]); 3) folate, a ligand bound by the folate receptor on cells of cancers that include cancers of the ovary, cervix, endometrium, lung, kidney, brain, breast, colon, and head and neck cancers [10].

The targeted moiety and the ligand can be directly conjugated through such means as reaction between the isothiocyanate group of FITC and free amine group of small ligands (e.g. folate, DUPA and CCK2R ligand). However, the use of a linking domain to connect the two molecules can be helpful as it can provide flexibility and stability to the SMC depending on the identity of the components comprising the SCM. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 pM or 10 pM, even more preferably at least about 100 pM.

The skilled artisan will understand and recognize that various means can be used to prepare SMCs comprised of a targeted moiety, a linking domain, and a ligand. Examples are provided in the Examples included herein.

Prior to being administered to a subject, the SCMs are prepared in a pharmaceutically acceptable formulation. Such formulations may contain a pharmaceutically acceptable carrier or diluent.

Exemplary SCMs included within the scope of the invention include the following molecules.

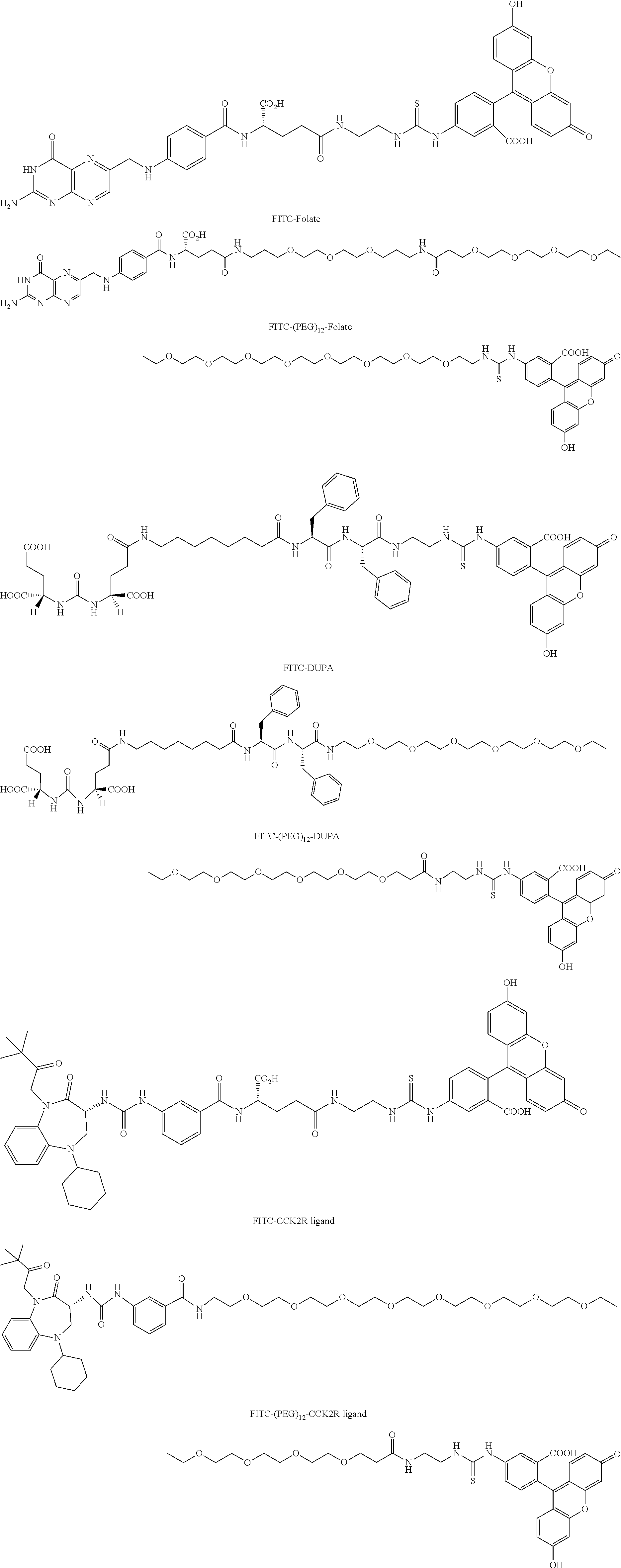

### Chimeric Antigen Receptors (CARs)

The CAR system of the present invention also utilizes cytotoxic lymphocytes engineered to express chimeric antigen receptors (CARs) that recognize and bind the targeting moiety of the SCMs. The CARs used in the CAR system comprise three domains. The first domain is the recognition region which, as the name suggests, recognizes and binds the targeting moiety. The second domain is the co-stimulation domain which enhances the proliferation and survival of the lymphocytes. The third domain is the activation signaling domain which is a cytotoxic lymphocyte activation signal. The three domains, together in the form of a fusion protein, comprise the CARs of the present invention.

As suggested above, the recognition region is the portion of a CAR that recognizes and binds a targeting moiety. The recognition regions comprising the CARs of the present invention are single chain fragment variable (scFv) regions of antibodies that bind the targeted moiety. Preferably, the scFv regions bind the targeted moiety with specificity. The identity of the antibody used in the production of the recognition region is limited only in that it binds the targeted moiety of the SCM. Thus, as non-limiting examples, scFv regions of antibodies that bind one of the following targeted moieties are included within the scope of the invention: DUPA, CCK2R ligand, folate. The scFv regions can be prepared from (i) antibodies known in the art that bind a targeted moiety, (ii) antibodies newly prepared using a selected targeted moiety as a hapten, and (iii) sequence variants derived from the scFv regions of such antibodies, e.g., scFv regions having at least about 80% sequence identity to the amino acid sequence of the scFv region from which they are derived. The use of unaltered (i.e., full size) antibodies, such as IgG, IgM, IgA, IgD or IgE, in the CAR or as the CAR is excluded from the scope of the invention.

The co-stimulation domain serves to enhance the proliferation and survival of the cytotoxic lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include: 1) CD28 [11]; 2) CD137 (4-1BB), a member of the tumor necrosis factor (TNF) receptor family [12]; 3) CD134 (OX40), a member of the TNFR-superfamily of receptors [13]; 4) CD278 (ICOS), a CD28-superfamily co-stimulatory molecule expressed on activated T cells [14]. The skilled artisan will understand that sequence variants of these noted co-stimulation domains can be used without adversely impacting the invention, where the variants have the same or similar activity as the domain on which they are modeled. Such variants will have at least about 80% sequence identity to the amino acid sequence of the domain from which they are derived.

In some embodiments of the invention, the CAR constructs comprise two co-stimulation domains. While the particular combinations include all possible variations of the four noted domains, specific examples include: 1) CD28+CD137 (4-1BB) and 2) CD28+CD134 (OX40).

The activation signaling domain serves to activate cytotoxic lymphocytes upon binding of the CAR to a targeted moiety. The identity of the activation signaling domain is limited only in that it has the ability to induce activation of the selected cytotoxic lymphocyte upon binding of the targeted moiety by the CAR. Suitable activation signaling domains include the T cell CD3ζ chain and Fc receptor γ. The skilled artisan will understand that sequence variants of these noted activation signaling domains can be used without adversely impacting the invention, where the variants have the same or similar activity as the domain on which they are modeled. Such variants will have at least about 80% sequence identity to the amino acid sequence of the domain from which they are derived.

Constructs encoding the CARs of the invention are prepared through genetic engineering. As an example, a plasmid or viral expression vector can be prepared that encodes a fusion protein comprising a recognition region, one or more co-stimulation domains, and an activation signaling domain, in frame and linked in a 5′ to 3′ direction. However, the CARs of the present invention are not limited in this arrangement and other arrangements are acceptable and include: (i) a recognition region, an activation signaling domain, and one or more co-stimulation domains, and (ii) a recognition region, a co-stimulation domain, and an activation signaling domain, linked in a 5′ to 3′ direction. It will be understood that because the recognition region must be free to bind the targeted moiety, the placement of the recognition region in z,999 fusion protein will generally be such that display of the region on the exterior of the cell is achieved. In the same manner, because the co-stimulation and activation signaling domains serve to induce activity and proliferation of the cytotoxic lymphocytes, the constructs will generally encode a fusion protein that displays these two domains in the interior of the cell.

The CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

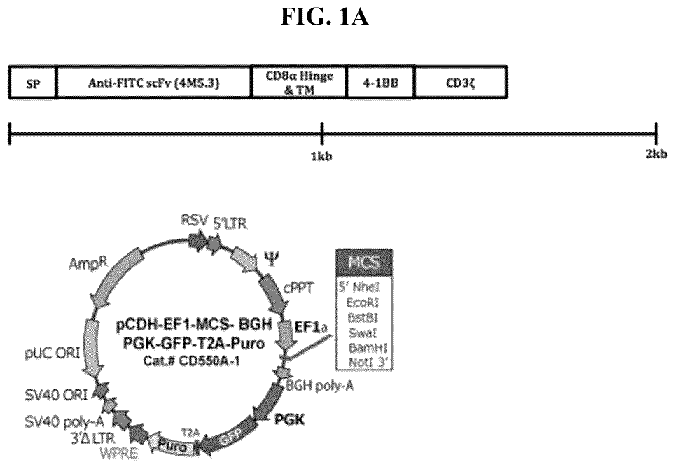

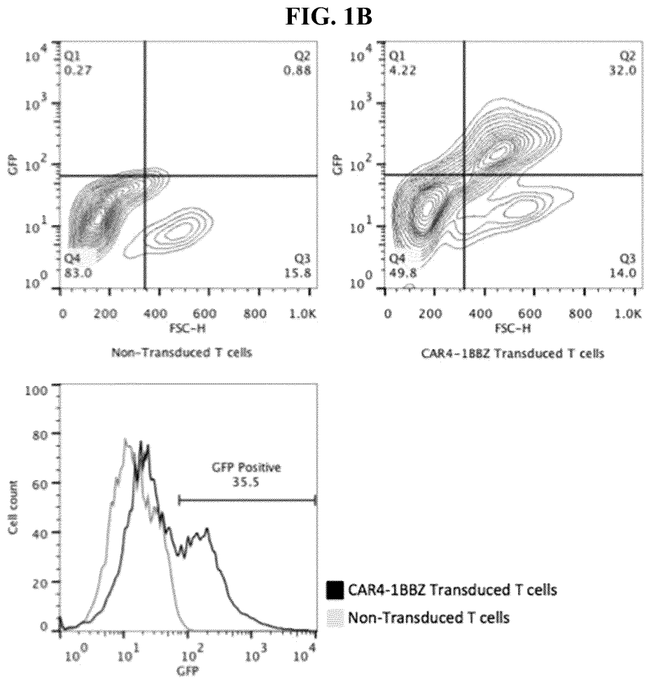

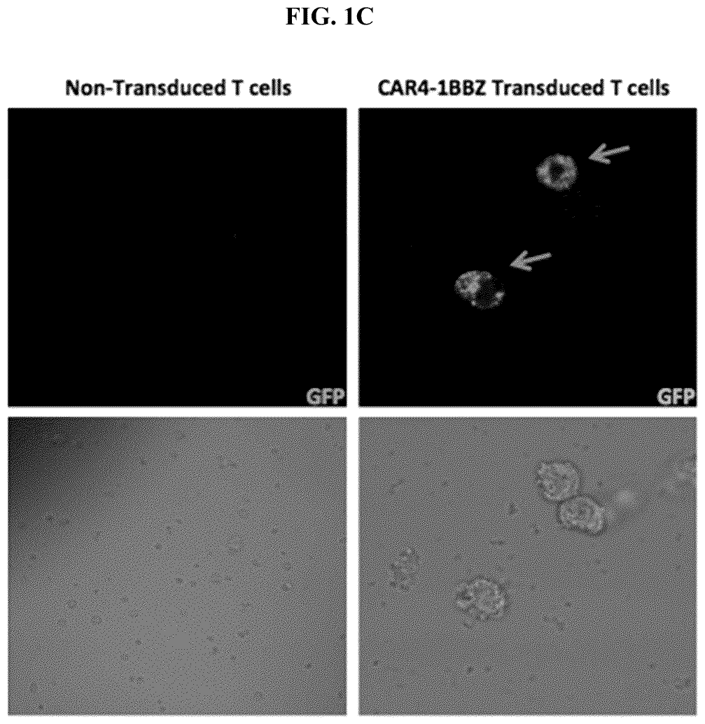

An example of an exemplary CAR of the present invention is shown in FIG. 1A where the fusion protein is encoded by a lentivirus expression vector and where “SP” is a signal peptide, the CAR is an anti-FITC CAR, a CD8α hinge is present, a transmembrane domain is present (“TM”), the co-stimulation domain is 4-1BB, and the activation signaling domain is CD3ζ. The sequence of the CAR-encoding vector is provided as SEQ ID NO:1.

In addition to the use of plasmid and viral vectors, cytotoxic lymphocytes can be engineered to express CARs of the invention through retrovirus, lentivirus (viral mediated CAR gene delivery system), sleeping beauty, and piggyback (transposon/transposase systems that include a non-viral mediated CAR gene delivery system).

While the affinity at which the CARs, expressed by the cytotoxic lymphocytes, bind to the targeted moiety can vary, and in some cases low affinity binding may be preferable (such as about 50 nM), the binding affinity of the CARs to the targeted ligand will generally be at least about 100 nM, 1 pM, or 10 pM, preferably at least about 100 pM, 1 fM or 10 fM, even more preferably at least about 100 fM.

### CAR-expressing Cytotoxic Lymphocytes

The cells used in the CAR system of the present invention are cytotoxic lymphocytes selected from (i) cytotoxic T cells (also variously known as cytotoxic T lymphocytes, CTLs, T killer cells, cytolytic T cells, CD8⁺ T cells, and killer T cells), natural killer (NK) cells, and lymphokine-activated killer (LAK) cells. Upon activation, each of these cytotoxic lymphocytes triggers the destruction of target tumor cells. For example, cytotoxic T cells trigger the destruction of target tumor cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target tumor cells.

The cytotoxic lymphocytes will preferably be autologous cells, although heterologous cells can also be used, such as when the subject being treated using the CAR system of the invention has received high-dose chemotherapy or radiation treatment to destroy the subject's immune system. Under such circumstances, allogenic cells can be used.

The cytotoxic lymphocytes can be isolated from peripheral blood using techniques well known in the art, include Ficoll density gradient centrifugation followed by negative selection to remove undesired cells.

Cytotoxic lymphocytes can be engineered to express CAR constructs by transfecting a population of lymphocytes with an expression vector encoding the CAR construct. Appropriates means for preparing a transduced population of lymphocytes expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples.

Transduced cytotoxic lymphocytes are grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma.

Prior to being administered to a subject, the cells are pelleted, washed, and resuspended in a pharmaceutically acceptable carrier or diluent. Exemplary formulations comprising CAR-expressing cytotoxic lymphocytes include formulations comprising the cells in sterile 290 mOsm saline, infusible cryomedia (containing Plasma-Lyte A, dextrose, sodium chloride injection, human serum albumin and DMSO), 0.9% NaCl with 2% human serum albumin or any other sterile 290 mOsm infusible materials.

### Methods of Treatment

The CAR system of the present invention can be used in the treatment of a subject having cancer. The methods of treatment encompassed by the invention generally includes the steps of (i) obtaining a population of autologous or heterologous cytotoxic lymphocytes, (ii) culturing the lymphocytes under conditions that promote the activation of the cells, (iii) transfecting the lymphocytes with an expression vector encoding a CAR, (iv) administering a formulation comprising the transfected lymphocytes to a subject having cancer, and (v) administering a formulation comprising SCM to the subject.

The invention also includes variations on this theme such, as administering the formulation comprising SMC to the subject before the formulation comprising the transfected lymphocytes, or at the same time as the formulation comprising the transfected lymphocytes. A further variation includes culturing the formulation comprising the transfected lymphocytes with the SCM prior to administration to the subject.

The population of cytotoxic lymphocytes can be obtained from a subject by means well known in the art. For example, cytotoxic T cells can be obtained by collecting peripheral blood from the subject, subjecting the blood to Ficoll density gradient centrifugation, and then using a negative T cell isolation kit (such as EasySep™ T Cell Isolation Kit) to isolate a population of cytotoxic T cells from the blood. While the population of cytotoxic lymphocytes need not be pure and may contain other blood cells such as T cells, monocytes, macrophages, natural killer cells and B cells, depending of the population being collected, preferably the population comprises at least about 90% of the selected cell type. In particular aspects, the population comprises at least about 90%, 91%, 92%, 93%, 94%, 95%, 96%, 97%, 98%, 99%, or even 100% of the selected cell type. As indicated earlier, the population of cells may come from the subject to be treated, from one or more different subjects, or the population may be a combination of cells from the subject to be treated and one or more different subjects.

After the population of cytotoxic lymphocytes is obtained, the cells are cultured under conditions that promote the activation of the cells. The culture conditions will be such that the cells can be administered to a subject without concern for reactivity against components of the culture. For example, when the population will be administered to a human, the culture conditions will not include bovine serum products, such as bovine serum albumin. The activation of the lymphocytes in the culture can be achieved by introducing known activators into the culture, such as anti-CD3 antibodies in the case of cytotoxic T cells. Other suitable activators include anti-CD28 antibodies. The population of lymphocytes will generally be cultured under conditions promoting activation for about 1 to 4 days. The appropriate level of cellular activation can be determined by cell size, proliferation rate or activation markers by flow cytometry.

After the population of cytotoxic lymphocytes has been cultured under conditions promoting activation, the cells are transfected with an expression vector encoding a CAR. Such vectors are described above, along with suitable means of transfection. After transfection, the resulting population of cells can be immediately administered to a subject or the cells can be culture for at least about 1, 2, 3, 4, 5, 6, 7, 8, 9, 10, 11, 12, 13, 14, 15, 16, 17, 18 or more days, or between about 5 and 12 days, between about 6 and 13 days, between about 7 and 14 days, or between about 8 and 15 days, for example, to allow time for the cells to recover from the transfection. Suitable culture conditions with be the same as those conditions under which the cells were culture while activation was being promoted, either with or without the agent that was used to promote activation and expansion.

When the transfected cells are ready a formulation comprising the cells is prepared and administered to a subject having cancer. Prior to administration, the population of cells can be washed and resuspended in a pharmaceutically acceptable carrier or diluent to form the formulation. Such carriers and diluents include, but are not limited to, sterile 290 mOsm saline, infusible cryomedia (containing Plasma-Lyte A, dextrose, sodium chloride injection, human serum albumin and DMSO), 0.9% NaCl with 2% human serum albumin or any other sterile 290 mOsm infusible materials. Alternatively, depending on the identity of the culture media used in the previous step, the cells can be administered in the culture media as the formulation, or concentrated and resuspended in the culture media before administration. The formulation can be administered to the subject via suitable means, such as parenteral administration, e.g., intradermally, subcutaneously, intramuscularly, intraperitoneally, intravenously, or intrathecally.

The total number of cells and the concentration of cells in the formulation administered to a subject will vary depending on a number of factors including the type of cytotoxic lymphocytes being used, the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. However, suitable formulations comprising transduced lymphocytes include those having a volume of between about 5 ml and 200 ml, containing from about 1×10⁵ to 1×10¹⁵ transduced cells. Typical formulations comprise a volume of between about 10 ml and 125 ml, containing from about 1×10⁷ to 1×10¹⁰ transduced cells. An exemplary formulation comprises about 1×10⁹ transduced cells in a volume of about 100 ml.

The final step in the method is the administration of a formulation comprising SCM to the subject. As described above, the SCM will be prepared in a formulation appropriate for the subject receiving the molecules. The concentration of SCM in a SCM formulation will vary depending on factors that include the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. However, suitable formulations comprising SCM include those having a volume of between about 1 ml and 50 ml and contain between about 20 ug/kg body weight and 3 mg/kg body weight SCM. Typical formulations comprise a volume of between about 5 ml and 20 ml and contain between about 0.2 mg/kg body weight and 0.4 mg/kg body weight SCM. An exemplary formulation comprises about 50 ug/kg body weight SCM in a volume of about 10 ml.

The timing between the administration of transduced lymphocyte formulation and the SCM formation may range widely depending on factors that include the type of cytotoxic lymphocytes being used, the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. Indeed, the SCM formation may be administered prior to, simultaneous with, or after the lymphocyte formulation. In general, the SCM formation will be administered after the lymphocyte formulation, such as within 3, 6, 9, 12, 15, 18, 21, or 24 hours, or within 0.5, 1, 1.5, 2, 2.5, 3, 4 5, 6, 7, 8, 9, 10 or more days. When the SCM formation is administered before the lymphocyte formulation, the lymphocyte formulation will generally be administered within about 0.25, 0.5, 0.75, 1, 2, 3, 4, 5, 6, 7, 8, 9, 10, 11, 12 or more hours. When the SCM formation and the lymphocyte formulation are added simultaneously, it is preferable that the formations are not combined and thus administered separately to the subject.

Depending on the cancer being treatment the step of administering the lymphocyte formulation, or the step of administering the SCM formulation, or both, can be repeated one or more times. The particular number and order of the steps is not limited as the attending physician may find that a method can be practiced to the advantage of the subject using one or more of the following methodologies, or others not named here: (i) administering the lymphocyte formulation (A) followed by the SCM formulation (B), i.e., A then B; (ii) B then A; (iii) A then B then A then B; (iv) A then B then A; (v) B then A then B then A; (vi) A then A then B; (vii) B then A then A; (vii) B then B then A.

The formulations can be administered as single continuous doses, or they can be divided and administered as a multiple-dose regimen depending on the reaction (i.e., side effects) of the patient to the formulations.

The types of cancers which may be treated using the methods of the invention will be governed based on the identity of the ligand used in the SCM. When the ligands defined above are used (i.e., DUPA, CCK2R ligand, folate) cancers that may be treated using the CAR system and methods of the present invention generally include solid tumors, and more specifically include prostate cancer adenocarcinoma, hepatoma, colorectal liver metastasis, and cancers of neuroendocrine origin.

In each of the embodiments and aspects of the invention, the subject is a human, a non-human primate, bird, horse, cow, goat, sheep, a companion animal, such as a dog, cat or rodent, or other mammal.

### III. Examples

### 1. Generation of CAR4-1BBZ and Transduction of Mouse T Cells to Express CAR

To generate modified T cells containing CAR that target cancer cells by FITC-ligand small molecule conjugates, CAR constructs were designed and generated as shown in FIG. 1.

### A) Generation of Chimeric Antigen Receptor (CAR) in Lentiviral Vector

An overlap PCR method was used to generate CAR constructs comprising scFv against FITC (CAR4-1BBZ). scFV against FITC, 4M5.3 (Kd=270 fM, 762 bp) derived from anti-fluorescence (4-4-20) antibody, was synthesized based on a previous report [15]. As shown in FIG. 1, sequence encoding the mouse CD8α signal peptide (SP, 81 bp), the hinge and transmembrane region (207 bp), the cytoplasmic domain of 4-1BB (CD137, 144bp) and the CD3ζ chain (339 bp) were fused with the anti-FITC scFV by overlapping PCR. The resulting CAR construct (CAR4-1BBZ) (1533 bp; SEQ ID NO:2) was inserted into NheI/NotI cleaved lentiviral expression vector pCDH-EF1-MCS-BGH-PGK-GFP-T2A-Puro (System Biosciences, Mountain View, CA). CAR4-1BBZ expression is regulated by EF1α promoter in the lentiviral vector. The sequence of CAR constructs in lentiviral vector (CAR4-1BBZ) was confirmed by DNA sequencing (Purdue Genomic Core Facility) and is provided in SEQ ID NO:1. copGFP expression encoded in the lentiviral vector was monitored to identify CAR4-1BBZ expression (FIG. 1C).

B) Isolation and Transduction of Mouse T cells

T cells were isolated from mouse spleen or peripheral blood. To isolate T cells, mouse splenocytes and peripheral blood mononuclear cells (PBMC) were isolated by Ficoll density gradient centrifugation (GE Healthcare Lifesciences). After washing away remaining Ficoll solution, T cells were isolated by EasySep™ Mouse T Cell Isolation Kit (STEM CELL technologies). Purified T cells are cultured in RPMI 1640 with 10% heat inactivated fetal bovine serum (FBS), 1% penicillin and streptomycin sulfate, 10 mM HEPES. To prepare lentiviral virus containing CAR4-1BBZ, 293TN packaging cell line was co-transfected with CAR4-1BBZ lentiviral vector and packaging plasmids. After 48 and 72 hours transfection, supernatants containing CAR4-1BBZ lentivirus were harvested and virus particles were concentrated for transduction. For transduction of mouse T cells, isolated T cells were activated with Dynabeads coupled with anti-CD3/CD28 antibodies (Life Technologies) for 12-24 hours in the presence of mouse IL-2 (50 units/ml), then infected with lentiviral expression vector containing CAR4-1BBZ. Mouse IL-2 (50 units/ml) was provided every other day. After 96 hours, cells were harvested and the expression of CAR on transduced T cells was identified by flow cytometry. As shown in FIG. 1B, approximately 30% of transduced T cells expressed CAR4-1BBZ.

### C) Flow Cytometry

The expression of CAR4-1BBZ on transduced T cells was determined by flow cytometry. Since lentiviral expression backbone also encodes copGFP expression, CAR4-1BBZ transduced T cells were verified by copGFP expression. FIG. 1C shows the expression of CAR4-1BBZ on transduced T cells. Transduced T cells were further confirmed by confocal microscope. copGFP was expressed on positive transduced T cells containing CAR4-1BBZ (arrows). However, copGFP expression was not detected on non-transduced T cells that do not express CAR4-1BBZ. Data was analyzed with FlowJo software.

Based on these result, it was evident that that the CAR4-1BBZ constructs was successfully transduced to mouse T cells and that the transduced T cells express CAR4-1BBZ.

### D) Cell Culture

Two cancer cell lines were used in this study: L1210A and KB. L1210A is a murine leukemia cell line. KB is a human epidermoid carcinoma cell line. Both of them have higher folate receptor expression on cell surface.

L1210A and KB cells were cultured in folic acid-deficient RPMI medium, 10% of heat inactivated fetal bovine serum (FBS), 1% penicillin and streptomycin sulfate were included in the culture media.

### 2. Generation of Small Conjugate Molecules Comprising FITC and Ligands

### A) Synthesis of FITC-Folate

Folate-γ-ethylenediamine was coupled to FITC isomer I (Sigma-Aldrich, St. Louis, MO) in anhydrous dimethylsulfoxide in the presence of tetramethylguanidine and diisopropylamine. The crude product was loaded onto an Xterra RP18 preparative HPLC column (Waters) and eluted with gradient conditions starting with 99% 5 mM sodium phosphate (mobile phase A, pH7.4) and 1% acetonitrile (mobile phase B) and reaching 90% A and 10% B in 10 min at a flow rate of 20 mL/min. Under these conditions, the folate-FITC main peak typically eluted at 27-50 min. The quality of folate-FITC fraction was monitored by analytical reverse-phase HPLC with a UV detector. Fractions with greater than 98.0% purity (LCMS) were lyophilized to obtain the final folate-FITC product.

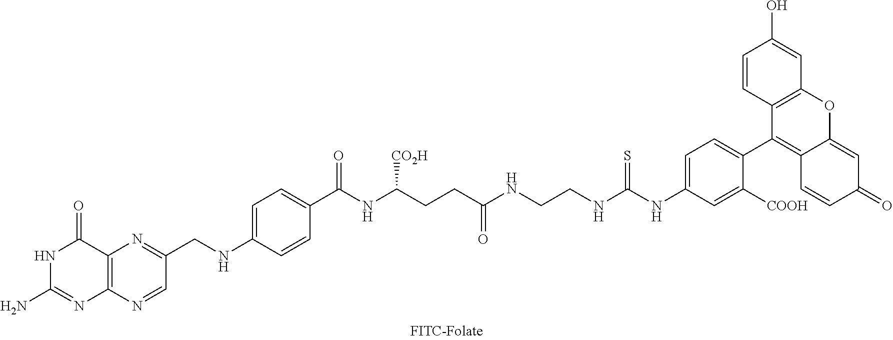 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

### B) Synthesis of FITC-(PEG)₁₂-Folate

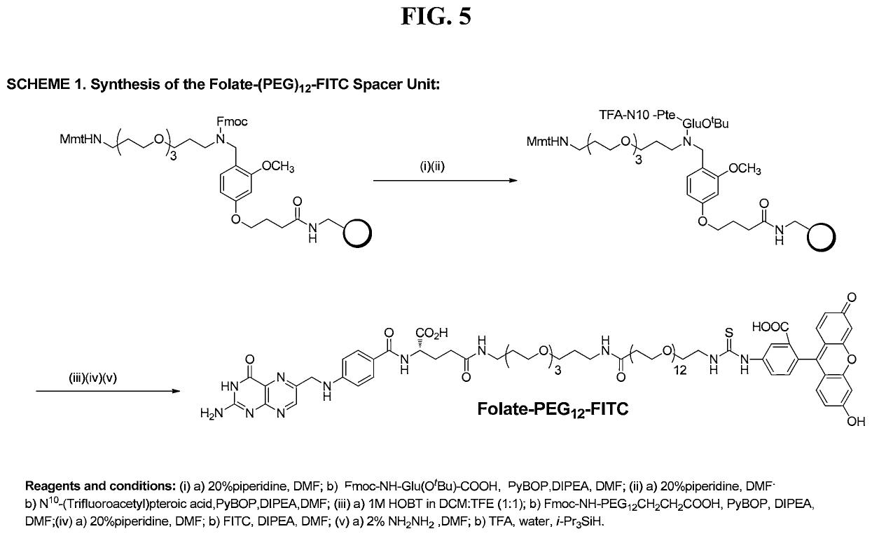

Universal PEG Nova Tag™ resin (0.2 g) was loaded into a peptide synthesis vessel and washed with i-PrOH (3×10 mL), followed by DMF (3×10 mL). Fmoc deprotection was carried out using 20% piperidine in DMF (3×10 mL). Kaiser tests were performed to assess reaction completion. To the vessel was then introduced a solution of Fmoc-Glu-(O-t-Bu)-OH (23.5 mg) in DMF, i-Pr₂NEt (4 equiv), and PyBOP (2 equiv). Fmoc deprotection was carried out using 20% piperidine in DMF (3×10 mL). To the vessel was then introduced a solution of N10-TFA-Pte-OH (22.5 mg), DMF, i-Pr₂NEt (4 equiv), and PyBOP (2 equiv). Argon was bubbled for 2 h, and resin was washed with DMF (3×3 mL) and i-PrOH (3×3 mL). After swelling the resin in DCM, a solution of IM HOBT in DCM/TFE (1:1) (2×3 mL) was added for removal of Mmt group. Argon was bubbled for 1 h, the solvent was removed and resin was washed with DMF (3×3 mL) and i-PrOH (3×3 mL). After swelling the resin in DMF, a solution of Fmoc-NH-(PEG)₁₂-COOH (46.3 mg) in DMF, i-Pr₂NEt (4 equiv), and PyBOP (2 equiv) was added. Argon was bubbled for 2 h, and resin was washed with DMF (3×3 mL) and i-PrOH (3×3 mL). Fmoc deprotection was carried out using 20% piperidine in DMF (3×10 mL). Kaiser tests were performed to assess reaction completion. To the vessel was then introduced a solution of FITC (21.4 mg) in DMF, i-Pr₂NEt (4 equiv), then Argon was bubbled for 2 h, and resin was washed with DMF (3×3 mL) and i-PrOH(3×3 mL). Then to the vessel was added 2% NH₂NH₂ in DMF (2×2 mL). Final compound was cleaved from resin using a TFA:H₂O:TIS (95:2.5:2.5) and concentrated under vacuum. The concentrated product was precipitated in Et₂O and dried under vacuum. The crude product was purified by using preparative RP-HPLC (mobile phase: A=10 mM ammonium acetate pH=7, B=ACN; method: 0% B to 30% B in 30 min at 13 mL/min). The pure fractions were pooled and freeze-dried, furnishing the FITC-(PEG)₁₂-Folate. FIG. 5 shows the synthetic scheme.

### C) Synthesis of FITC-(PEG)₁₂-DUPA

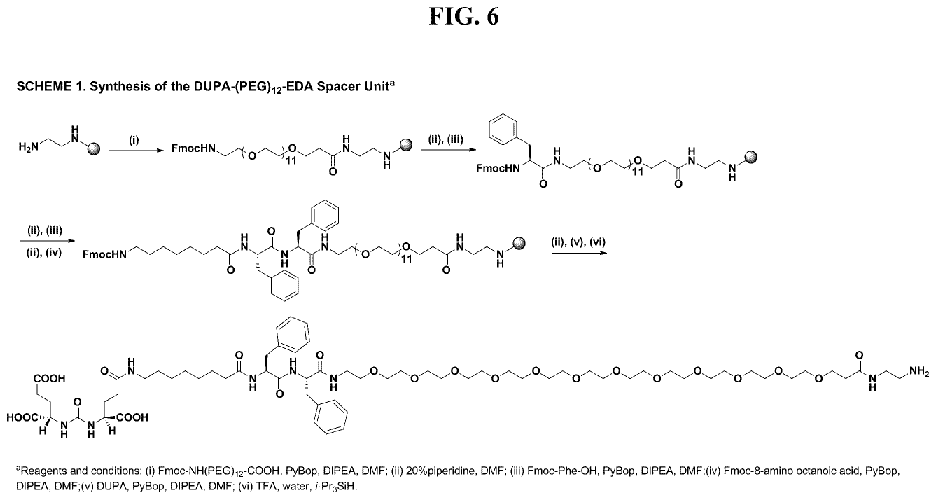

Synthesis of DUPA-(PEG)₁₂-EDA: 1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH-(PEG)₁₂-COOH (42.8 mg) in DMF, i-Pr₂NEt (2.5 equiv), and PyBOP (2.5 equiv). The resulting solution was bubbled with Ar for 1 h, the coupling solution was drained, and the resin washed with DMF (3×10 mL) and i-PrOH (3×10 mL). Kaiser tests were performed to assess reaction completion. Fmoc deprotection was carried out using 20% piperidine in DMF (3×10 mL). This procedure was repeated to complete the all coupling steps (2×1.5 equiv of Fmoc-Phe-OH and 1.5 equiv of 8-aminooctanoic acid and 1.2 equiv of DUPA were used on each of their respective coupling steps). After the DUPA coupling, the resin was washed with DMF (3×10 mL) and i-PrOH (3×10 mL) and dried under reduced pressure. The peptide was cleaved from the resin in the peptide synthesis vessel using a cleavage mixture consisting of 95% CF₃CO₂H, 2.5% H₂O, and 2.5% triisopropylsilane. Fifteen milliliters of the cleavage mixture was added to the peptide synthesis vessel, and the reaction was bubbled under Ar for 15 min. The resin was treated with two additional 10 mL quantities of the cleavage mixture for 5 min each. The cleavage mixture was concentrated to ca. 5 mL, and ethyl ether was added to induce precipitation. The precipitate was collected by centrifugation, washed with ethyl ether three times, and dried under high vacuum, resulting in the recovery of DUPA-(PEG)₁₂-EDA as crude material. FIG. 6 shows the synthetic scheme.

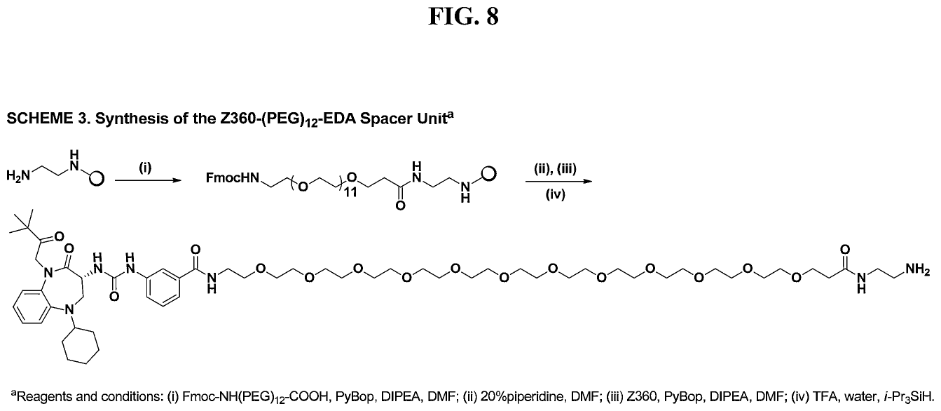

Synthesis of FITC-(PEG)₁₂-DUPA: To a stirred solution of the crude DUPA-(PEG)₁₂-EDA (10 mg) and FITC (5.6 mg) in dimethylsulfoxide (DMSO, 1 mL) was added Pr₂NEt (5 equiv) at room temperature and stirring continued for 6 hr under argon. The reaction was monitored by LCMS and purified by preparative HPLC (mobile phase: A=10 mM ammonium acetate pH=7, B=ACN; method: 0% B to 50% B in 30 min at 13 mL/min). The pure fractions were pooled and freeze-dried, furnishing the FITC-(PEG)₁₂-DUPA. FIG. 8 shows the synthetic scheme.

### D) Synthesis of FITC-(PEG)₁₂-CCK2R Ligand (Z360)

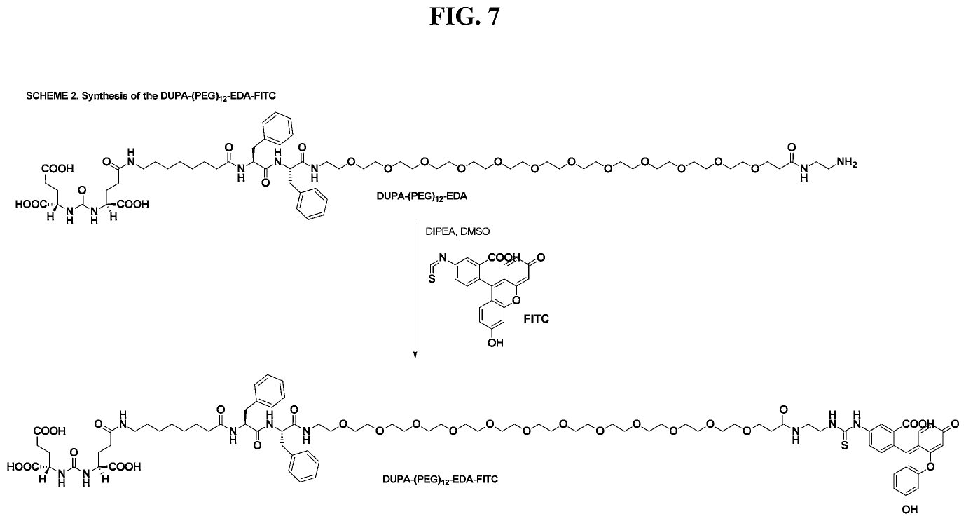

Synthesis of CCK2R ligand-(PEG)₁₂-EDA: 1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH-(PEG)₁₂-COOH (42.8 mg) in DMF, i-Pr₂NEt (2 equiv), and PyBOP (1 equiv). The resulting solution was bubbled with Ar for 1 h, the coupling solution was drained, and the resin washed with DMF (3×10 mL) and i-PrOH (3×10 mL). Kaiser tests were performed to assess reaction completion. Fmoc deprotection was carried out using 20% piperidine in DMF (3×10 mL). Then added Z360 (10 mg) in DMF, i-Pr₂NEt (2.5 equiv), and PyBOP (2.5 equiv). After the Z360 coupling, the resin was washed with DMF (3×10 mL) and i-PrOH (3×10 mL) and dried under reduced pressure. The peptide was cleaved from the resin in the peptide synthesis vessel using a cleavage mixture consisting of 95% CF₃CO₂H, 2.5% H₂O, and 2.5% triisopropylsilane. Fifteen milliliters of the cleavage mixture was added to the peptide synthesis vessel, and the reaction was bubbled under Ar for 15 min. The resin was treated with two additional 10 mL quantities of the cleavage mixture for 5 min each. The cleavage mixture was concentrated to ca. 5 mL, and ethyl ether was added to induce precipitation. The precipitate was collected by centrifugation, washed with ethyl ether three times, and dried under high vacuum, resulting in the recovery of crude material. FIG. 7 shows the synthetic scheme.

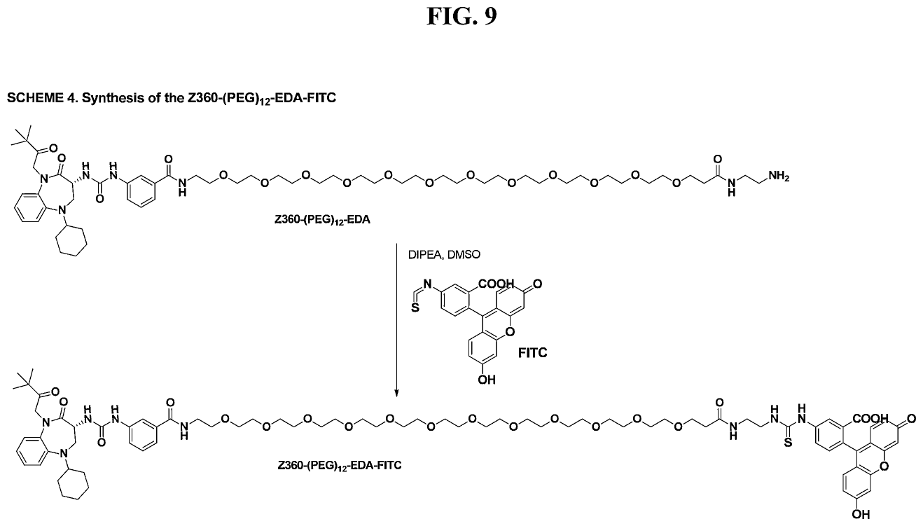

Synthesis of FITC-(PEG)₁₂-CCK2R ligand: To a stirred solution of the crude CCK2R ligand-(PEG)₁₂-EDA (10 mg) and FITC (6 mg) in dimethylsulfoxide (DMSO, 1 mL) was added Pr₂NEt (5 equiv) at room temperature and stirring continued for 6 hr under argon. The reaction was monitored by LCMS and purified by preparative HPLC (mobile phase: A=10 mM ammonium acetate pH=7, B=ACN; method: 0% B to 50% B in 30 min at 13 mL/min). The pure fractions were pooled and freeze-dried, furnishing the FITC-(PEG)₁₂-CCK2R ligand. FIG. 9 shows the synthetic scheme.

3. Binding of FITC-Folate and FITC-(PEG)₁₂-Folate Conjugates to Transduced T Cells Through Anti-FITC scFV in CAR4-1BBZ

To examine the ability of the transduced T cells containing CAR4-1BBZ to bind the FITC-Folate conjugates, binding assays with the FITC-Folate conjugate and the FITC-(PEG)₁₂-Folate conjugate were performed. Since the excitation wavelength of FITC in the FITC-Folate conjugates overlapped with copGFP in CAR4-1BB transduced T cells, it was difficult to distinguish between FITC binding and copGFP expression. Therefore, an anti-Folate Acid (FA) monoclonal antibody and a fluorephore (excitation 640 nm)-labeled anti-mouse IgG antibody were utilized.

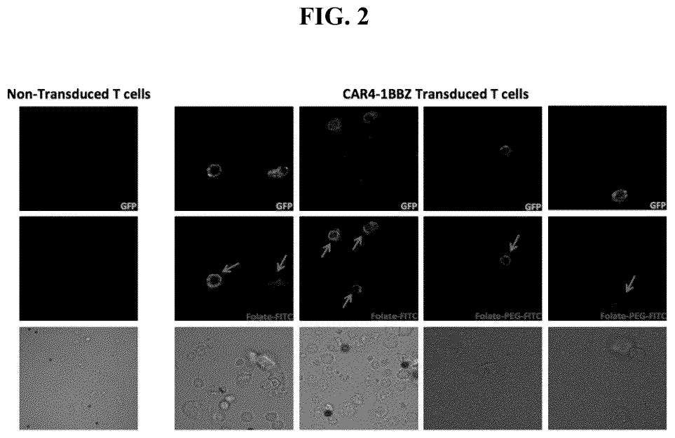

First, the transduced T cells were incubated with FITC-Folate or FITC-(PEG)₁₂-Folate conjugates at room temperature for 1 hour. After washing with 1× PBS, transduced T cells were incubated with anti-FA monoclonal antibody (1:15 dilution) for 1 hour. After another washing with 1× PBS, transduced T cells further were incubated with fluorophore (excitation 640 nm) labeled anti-mouse IgG antibody (1:50 dilution). Finally, unbound antibodies were washed away and a confocal microscope was used to confirm the FITC-Folate and FITC-(PEG)₁₂-Folate conjugates binding ability. All data was analyzed by Olympus Fluoview software. As shown in FIG. 2, copGFP expression was observed on the transduced T cells containing CAR 4-1BBZ (top row), but not in non-transduced T cells. As shown in the middle panel, FITC-folate binding was observed on CAR-1BBZ transduced T cells, but not on non-transduced T cells. More importantly, only those transduced T cells showing copGFP expression (top row) also show FITC-Folate conjugates binding (middle row). By confocal microscopy, it was confirmed that FITC-Folate and FITC-(PEG)₁₂-Folate conjugates were successfully bound to anti-FITC in transduced T cells, but not to non-transduced T cells. Simultaneously, binding of FITC-Folate and FITC-(PEG)₁₂-Folate conjugates (arrows) were also detected on the CAR4-1BBZ transduced T cells. Based on this data, it was concluded that transduced T cells express CAR4-1BBZ, and that FITC-Folate conjugates can bind to transduced T cells through anti-FITC scFv expressed by CAR4-1BBZ transduction.

### 4. The Binding of FITC-Folate Conjugates to FR Positive Cancer Cells

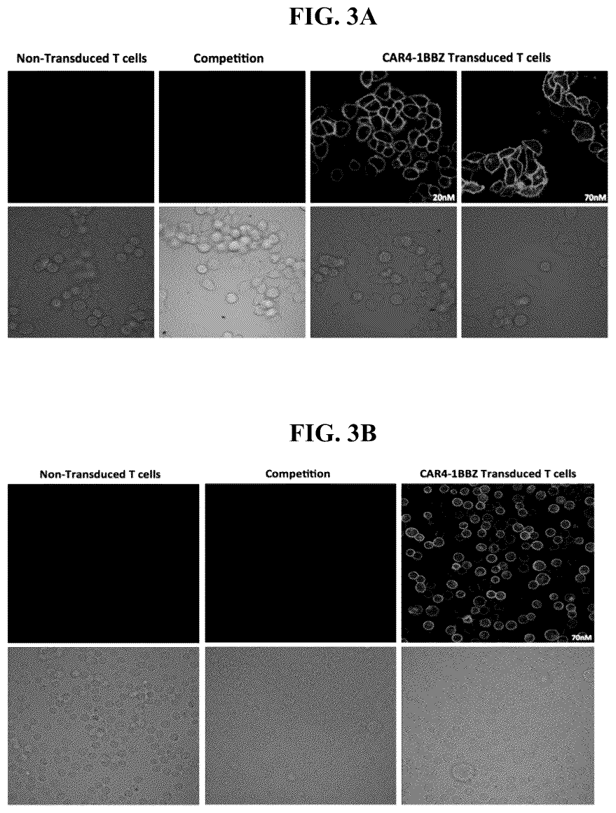

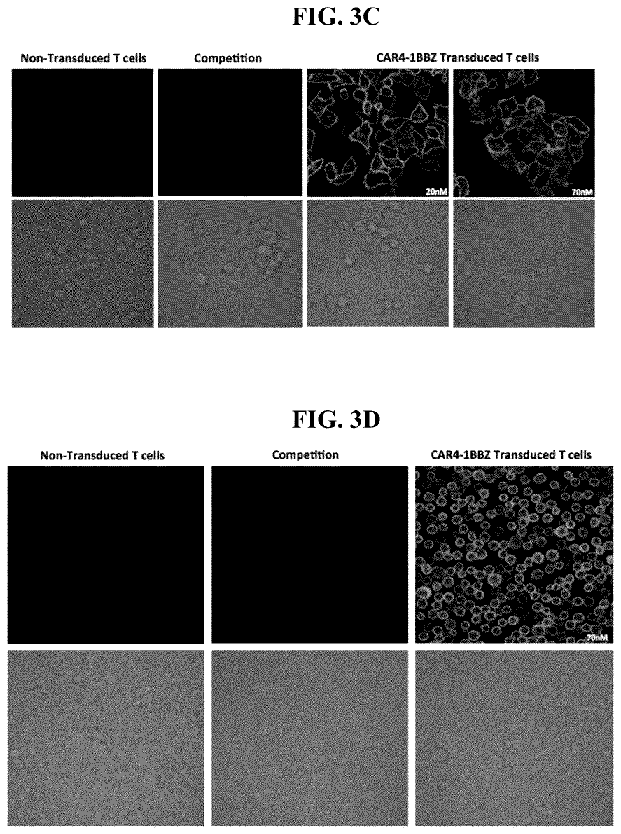

The ability of the FITC-Folate and FITC-(PEG)₁₂-Folate conjugates to bind folate receptor (FR) positive cancer cells was next investigated. FR positive cells lines L1210A (Mouse lymphocytic leukemia) and KB (Mouth epidermal carcinoma), which are FR positive cell lines, were used to test the binding affinity of the FITC-Folate conjugates. Briefly, 3-4×10⁴ cancer cells were prepared to perform binding affinity assays with FITC-Folate and FITC-(PEG)₁₂-Folate conjugates. With two different concentrations (e.g. 20 nM, 70 nM), the two FITC-Folate conjugates were incubated with cancer cells at room temperature for 1 hour. Since both FITC-Folate conjugates have fluorescence, the binding ability of FITC-Folate conjugates to cancer cells can be detected by florescence microscopy. After two washes with 1× phosphate buffered saline (PBS) to remove all unbound compound, the cancer cells were observed by confocal microscope. In order to specify whether FITC-Folate conjugates bind to cancer cells via FR on the surface of cancer cells, Folate Acid was also incubated with cancer cells as a competition compound. As shown in FIGS. 3A, 3B, 3C, and 3D, the FITC-Folate conjugates can bind to both cancer cells (KB, L1210A) via FR on the cell surface. As shown in competition panel, addition of 50× excess folate acid blocked FITC-Folate binding with these cancer cells, which indicates the specific binding is between folate acid and FR on cell surface.

As shown in FIG. 3A, the FITC-Folate conjugate was internalized into KB cell cytoplasm, caused by FR mediated endocytosis. However, as shown in FIG. 3C, the FITC-(PEG)₁₂-Folate conjugate, which has a (PEG)₁₂ linker between Folate and FITC, stayed on the extracellular surface of the KB cells.

From this data it was concluded that the FITC-Folate and FITC-(PEG)₁₂-Folate conjugates can bind to cancer cells through FR on the surface of cancer cells. It was also found that the PEG linker between Folate and FITC can prohibit the internalization of FITC-Folate conjugate. Surface localization of FITC-Folate conjugate would help increase the chance for cancer cells to be recognized by transduced T cell containing CAR4-1BBZ. If FITC-Folate conjugates are internalized via FR mediated endocytosis, transduced T cells cannot target cancer cells through FITC-Folate conjugates.

### 5. Cytotoxicity of Anti-FITC CAR-modified T Cells Against Folate Receptor Positive (FR+) Cancer Cells

In order to explore whether CAR-expressing T cells can kill cancer cells, ⁵¹Chromium release assays were conducted. In this assay, cancer cells are labeled with ⁵¹Cr. When cancer cells are lysed, ⁵¹Cr would be released from cancer cells to supernatant. By measuring ⁵¹Cr in supernatant, the number of cancer cells killed can be determined.

First, target cancer cells L1210A were incubated in 50 μL growth medium containing 50 μCi ⁵¹Chromium to get labeled. After washing with PBS ×3, L1210A cells were resuspended and incubated at 37° C. for 1 hour in the growth medium containing 70 nM FITC-Folate conjugate or FITC-(PEG)₁₂-Folate conjugate. After washing away excess FITC conjugates, 5×10³ target cancer cells were added in each well of 96-well plates. When adhesive KB cells were used as the target, they were treated similar as L1210A cells, except KB cells were grown in 96-well plates for 24 hours before ⁵¹Cr labeling and FITC-Folate conjugates binding were performed in 96-well plates. Effector T cells were then harvested, resuspended in growth media and added to the wells containing target cancer cells. The effector T cell to target cancer cell ratio was 20:1. After incubation for 4-10 hours, 20 μL aliquots of cell-free supernatant were harvested and ⁵¹Cr in the supernatants was measured with a scintillation counter or a [symbol] -counter.

Percent specific cytolysis was calculated using following formula:

%cytolysis=(Experimental ⁵¹Cr release−control ⁵¹Cr release)/(Maximum ⁵¹Cr release−control ⁵¹Cr release)×100

Control wells contained only target cancer cells, effector T cells were never added in these wells. Maximum ⁵¹Cr release was determined by measuring ⁵¹Cr release from labeled target cells treated with 2.5% SDS to lyse all cells.

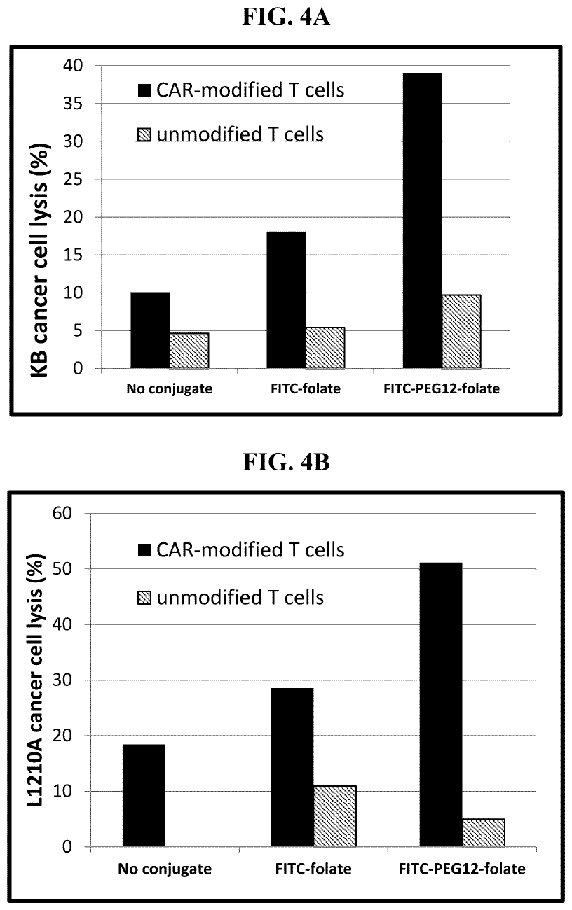

As shown in FIG. 4A, in the absence of FITC-Folate conjugates, CAR-expressing T cells showed negligible activity (˜5%) on target KB cell cytolysis. In the presence of FITC-Folate conjugate, CAR-expressing T cells showed ˜18% cancer cell cytolysis. It is implicated that FITC-Folate conjugate acts as a bridge to redirect anti-FITC CAR-expressing T cells to FR+KB cells. Furthermore, in the presence of FITC-(PEG)₁₂-Folate conjugate, which has a ˜40 Å (PEG)₁₂ spacer between FITC and Folate molecules, anti-FITC CAR-expressing T cells showed much higher activity (˜39%) on cancer cell cytolysis. With ˜40 Å distance between FITC and Folate, this conjugate can redirect CAR-expressing T cells to FR+KB cells much better (39% vs. 18%). As the non-specific cytotoxicity control, unmodified T cells showed only 5-10% cytolysis. The existence or absence of the FITC-Folate conjugates did not show any effect on unmodified T cell cytotoxicity against FR+KB cells.

FIG. 4B shows cytotoxicity of anti-FITC CAR modified T cells against cancer cell line L1210A. In the absence of FITC-Folate conjugates, CAR-expressing T cells showed negligible activity (2-3%) on target L1210A cell cytolysis. In the presence of FITC-Folate conjugate, CAR-expressing T cells showed ˜29% cancer cell cytolysis. It is implicated that FITC-Folate conjugate acts as a bridge to redirect anti-FITC CAR-expressing T cells to FR+L1210A cells. Furthermore, in the presence of the FITC-(PEG)₁₂-Folate conjugate, which has a ˜40 Å (PEG)₁₂ spacer between FITC and Folate molecules, anti-FITC CAR-expressing T cells showed much higher activity (˜51%) on FR+L1210A cytolysis. With ˜40 Å distance between FITC and Folate, this conjugate can redirect CAR-expressing T cells to FR+L1210A much better (51% vs. 29%). As the non-specific cytotoxicity control, unmodified T cells showed only 5-10% cytolysis as expected.

From this data it was concluded that anti-FITC CAR-expressing T cells do not have innate cytotoxicity against FR+cancer cells. However, in the presence of the FITC-Folate conjugate, anti-FITC CAR-expressing T cells are redirected to FR+cancer cells and cause specific cytolysis, and the activation of CAR-expressing T cells can be regulated by controlling of the addition of FITC-Folate conjugates. Further, the FITC-(PEG)₁₂-Folate conjugate, which has a ˜40 Å spacer between the FITC and the Folate molecules, can redirect anti-FITC CAR-expressing T cells to FR+cancer cells much better than the conjugate without the spacer.

While the invention has been described with reference to certain particular embodiments thereof, those skilled in the art will appreciate that various modifications may be made without departing from the spirit and scope of the invention. The scope of the appended claims is not to be limited to the specific embodiments described.

### References

All patents and publications mentioned in this specification are indicative of the level of skill of those skilled in the art to which the invention pertains. Each cited patent and publication is incorporated herein by reference in its entirety. All of the following references have been cited in this application:
1. Sadelain, M. et al., The basic principles of chimeric antigen receptor design. Cancer Discovery. 2013. 3(4):388-98.
2. Cartellieri, M. et al., Chimeric antigen receptor-engineered T cells for immunotherapy of cancer. J Biomedicine and Biotechnology. 2010. Article ID 956304, 13 pages.
3. Urba, W. J. et al., Redirecting T cells. N Engl J Med. 2011. 365:8.
4. Porter, D. L. et al., Chimeric antigen receptor-modified T cells in chronic lymphoid leukemia. N Engl J Med. 2011. 365:725-33.
5. Cor, H. J. et al., Treatment of Metastatic Renal Cell Carcinoma With Autologous T-Lymphocytes Genetically Retargeted Against Carbonic Anhydrase IX: First Clinical Experience. J Clin Oncol. 2006. 24(13):e20-2.
6. Kochenderfer, J. N. et al., B-cell depletion and remissions of malignancy along with cytokine-assoicated toxicity in clinical trial of anti-CD19 chimeric antigen receptor transduced T cells. Blood. 2012. 119(12):2790-20.
7. Reichert, J. M. Day 1, Emerging Disruptive Technologies and Cutting-Edge Analytical Techniques. MAbs 2009. 1(3):190-209.
8. Kularatne, S. A. et al., Prostate-specific membrane antigen targeted imaging and therapy of prostate cancer using a PSMA inhibitor as a homing ligand. Mol Pharm. 2009. 6(3):780-9.
9. Wayua. C. et al., Evaluation of a Cholecystokinin 2 Receptor-Targeted Near-Infrared Dye for Fluorescence-Guided Surgery of Cancer. Molecular Pharmaceutics. 2013. (ePublication).
10. Sega, E. I. et al., Tumor detection using folate receptor-targeted imaging agents. Cancer Metastasis Rev. 2008. 27(4):655-64.
11. Alvarez-Vallina, L. et al, Antigen-specific targeting of CD28-mediated T cell co-stimulation using chimeric single-chain antibody variable fragment-CD28 receptors. Eur J Immumol. 1996. 26(10):2304-9.
12. Imai, C. et al., Chimeric receptors with 4-1BB signaling capacity provoke potent cytotoxicity against acute lymphoblastic leukemia. Leukemia. 2004. 18:676-84.
13. Latza, U. et al., The human OX40 homolog: cDNA structure expression and chromosomal assignment of the ACT35 antigen. Eur. J. Immunol. 1994. 24:677.
14. Hutloff, A. et al., ICOS is an inducible T-cell costimulator structurally and functionally related to CD28. Nature. 1999. 397:263.
15. Orr, B. A., et al., Rapid Method for Measuring ScFv Thermal Stability by Yeast Surface Display. Biotechnol Prog. 2003. 19(2):631-8.
16. Kolmar, H. et al., Alternative binding proteins: biological activity and therapeutic potential of cysteine-knot miniproteins. The FEBS Journal. 2008. 275(11): 26684-90

1-48. (canceled)

49. A method of killing cancer cells, the method comprising:
(a) administering a therapeutically effective number of cytotoxic lymphocytes expressing a chimeric antigen receptor (CAR) that specifically binds to a targeted moiety; and
(b) administering a small conjugate molecule (SCM) comprising the targeted moiety conjugated to a tumor receptor ligand to the subject, wherein the tumor receptor ligand is recognized and bound by a receptor on the surface of a cell of the cancer,
thereby treating the cancer;
wherein the targeted moiety is a molecule selected from the group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PPP), tetrafluorophenyl ester (TFP), a knottin, a centyrin, and a DARPin; and
wherein the tumor receptor ligand is selected from the group consisting of folate, DUPA, and CCK2R ligand.

50. The method of claim 49, wherein the CAR is a fusion protein comprising a recognition region, a transmembrane domain, a co-stimulation domain, and an activation signaling domain, and wherein the recognition region of the CAR specifically binds to a targeted moiety.

51. The method of claim 50, wherein the recognition region of the CAR is a single chain fragment variable (scFv) region.

52. The method of claim 50, wherein the recognition region of the CAR specifically binds FITC.

53. The method of claim 50, wherein the recognition region of the CAR is a single chain fragment variable (scFv) region of an anti-FITC antibody.

54. The method of claim 50, wherein the co-stimulation domain of the CAR is selected from the group consisting of CD28, CD137 (4-IBB), CD134 (OX40), and CD278 (ICOS).

55. The method of claim 50, wherein the activation signaling domain of the CAR is the T cell CD3z chain or Fc receptor γ.

56. The method of claim 49, wherein the targeted moiety and the tumor receptor ligand are conjugated via a linker domain.

57. The method of claim 56, wherein the linker domain is selected from the group consisting of polyethylene glycol (PEG), polyproline, a hydrophilic amino acid, a sugar, an unnatural peptideoglycan, polyvinylpyrrolidone, and pluronic F-127.

58. The method of claim 49, wherein the conjugates are not endocytosed by receptors of the cancer cells.

59. The method of claim 49, wherein induction of cytokine storm is reduced.

60. The method of claim 49, wherein the method comprises transfecting cytotoxic lymphocytes with a vector encoding the CAR prior to contacting the cancer cells with the cytotoxic lymphocytes expressing the CAR.

61. The method of claim 60, wherein the vector is a lentiviral vector.

62. The method of claim 49, wherein the method causes lysis of at least about 18%, at least about 29%, or at least about 51% of the cancer cells.

63. The method of claim 49, wherein the cytotoxic lymphocytes are cytotoxic T lymphocytes.

64. The method of claim 49, wherein the cancer cells are from a cancer that is one or more of a cancer of the brain, thyroid, lung, pancreas, kidney, stomach, gastrointestinal stroma, endometrium, breast, cervix, ovary, colon, prostate, leukemias, lymphomas, other blood-related cancers, or head and neck cancer.

65. The method of claim 49, wherein the cancer cells are folate receptor expressing (FR+) cancer cells.

66. A two component cancer therapeutic comprising:
(a) a small conjugate molecule (SCM) comprising a targeted moiety conjugated to a tumor receptor ligand, wherein the tumor receptor ligand is selected from the group consisting of folate, DUPA, and CCK2R ligand; and
(b) chimeric antigen receptor (CAR)-expressing cytotoxic lymphocytes, wherein the CAR is a fusion protein comprising a recognition region, a co-stimulation domain and an activation signaling domain, and wherein the CAR has binding specificity for the targeted moiety or can be bound by the targeted moiety.

67. The two component cancer therapeutic of claim 66, wherein the targeted moiety is FITC.
